(19) United States
(12) Patent Application Publication
(10) Pub. No.: US 2024/0115237 A1
(43) Pub. Date: Apr. 11, 2024

(54) CONFIGURING ULTRASOUND SYSTEMS BASED ON SCANNER GRIP

(71) Applicant: FUJIFILM SONOSITE, INC., Bothell, WA (US)

(72) Inventors: Craig Chamberlain, Seattle, WA (US); Christopher Howard, Seattle, WA (US); Andrew Lundberg, Woodinville, WA (US); Davin Dhatt, Woodinville, WA (US)

(21) Appl. No.: 18/045,477

(22) Filed: Oct. 11, 2022

(51) Int. Cl. A61B 8/00; A61B 8/08; G01S 7/52; G01S 15/89

(52) U.S. Cl. CPC A61B 8/4455; A61B 8/0891; A61B 8/467; A61B 8/485; A61B 8/5223; A61B 8/54; G01S 7/52053; G01S 15/8934; A61B 8/461

(57) ABSTRACT

Systems and methods to configure ultrasound systems based on a scanner grip are described. In some embodiments, an ultrasound system includes an ultrasound scanner having a touch sensitive surface and a processor that is configured to determine a grip orientation on the touch sensitive surface; and activate, based on the grip orientation, a region of the touch sensitive surface to accept a user input.

Embodiments disclosed herein relate to ultrasound systems. More specifically, embodiments disclosed herein relate to configuring ultrasound systems based on a scanner grip.

### BACKGROUND

Ultrasound systems can generate ultrasound images by transmitting sound waves at frequencies above the audible spectrum into a body, receiving echo signals caused by the sound waves reflecting from internal body parts, and converting the echo signals into electrical signals for image generation. Because they are non-invasive and can provide immediate imaging results without delay, ultrasound systems are often used at the point of care, such as at the bedside, in an emergency department, at various types of care facilities, etc.

Conventional ultrasound systems require manual, explicit configuration by an operator. Currently, to operate an ultrasound system, an operator (e.g., a sonographer) is often required to devote significant resources to configure the ultrasound system, e.g., set imaging parameters, for use. For instance, the operator may need to set imaging parameters such as depth and gain, an imaging mode (e.g., B-mode vs. M-mode), an examination type, etc. In some cases, the operator is required to configure the ultrasound system in a certain way so that the operator can enter a bill for the ultrasound examination. For example, the operator may be required to configure the ultrasound system for an approved examination type for a given patient since the billing system in the care facility will not process bills for ultrasound examinations not of the approved examination type for the patient.

As operators of conventional ultrasound systems necessarily divert their attention away from the patient towards the ultrasound system, the patients may not receive the best care possible.

### SUMMARY

Systems and methods to configure ultrasound systems based on a scanner grip are described. In some embodiments, an ultrasound system includes an ultrasound scanner having a touch sensitive surface and a processor that is configured to determine a grip orientation on the touch sensitive surface and activate, based on the grip orientation, a region of the touch sensitive surface to accept a user input.

In some embodiments, an ultrasound system includes an ultrasound scanner having a touch sensitive surface. The ultrasound scanner is configured to generate ultrasound data based on reflections of ultrasound signals transmitted by the ultrasound scanner at an anatomy. The ultrasound system includes a display device that is configured to generate an ultrasound image of the anatomy based on the ultrasound data. The ultrasound system also includes a processor that is configured to determine locations of pressure on the touch sensitive surface and amounts of the pressure at the locations and determine, based on the locations and the amounts of the pressure and the ultrasound image, an elasticity of the anatomy.

In some embodiments, an ultrasound system includes an ultrasound scanner having a touch sensitive surface. The ultrasound scanner is configured to generate ultrasound data based on reflections of ultrasound signals transmitted by the ultrasound scanner. The ultrasound system includes a display device that is configured to display an ultrasound image that is based on the ultrasound data. The ultrasound system also includes a processor that is configured to determine a grip orientation on the touch sensitive surface and set, based on the grip orientation, an imaging parameter for at least one of the display device and the ultrasound scanner.

In some embodiments, a method implemented by a computing device to determine an anatomy being imaged includes determining finger positions on an ultrasound scanner, determining an orientation of the ultrasound scanner, and determining, based on the finger positions and the orientation, the anatomy being imaged.

In some embodiments, a method implemented by a computing device includes determining a grip orientation on an ultrasound scanner. The grip orientation includes finger locations on a surface of the ultrasound scanner. The method also includes enabling, based on the finger locations, an active area on the surface of the ultrasound scanner. The method also includes receiving a touch input via the active area and controlling, based on the touch input, an object in an augmented or virtual reality environment.

In some embodiments, a method implemented by a computing device to image an anatomy includes determining finger positions on an ultrasound scanner, and determining an orientation of the ultrasound scanner. The method also includes configuring, based on the finger positions and the orientation, the computing device to image the anatomy.

Other systems, machines, and methods for handset wireless network connectivity are also described.

### BRIEF DESCRIPTION OF THE DRAWINGS

The appended drawings illustrate examples and are, therefore, exemplary embodiments and not considered to be limiting in scope.

FIG. 1A is a view illustrating an ultrasound system to detect a grip orientation according to some embodiments.

FIG. 1B is a view illustrating an ultrasound system that generates one or more grip maps to detect a grip orientation according to some embodiments.

FIG. 2 is a view illustrating an example use of grip maps that represent a grip orientation according to some embodiments.

FIG. 3 illustrates a method implemented by a computing device for controlling an object in an augmented reality (AR)/virtual reality (VR) environment based on a scanner grip orientation according to some embodiments.

FIG. 4 illustrates a method for configuring a device (e.g., an ultrasound machine or a computing device coupled to a scanner, such as a tablet) to image an anatomy based on a scanner grip orientation according to some embodiments.

FIG. 5 illustrates a method to configure an ultrasound system based on a scanner grip according to some embodiments.

FIG. 6 illustrates a method to determine an elasticity of an anatomy according to some embodiments.

FIG. 7 illustrates a method to set an imaging parameter for an ultrasound system according to some embodiments.

FIG. 8 is a block diagram of an example computing device that can perform one or more of the operations described herein, in accordance with some embodiments.

### DETAILED DESCRIPTION

Systems and methods to configure ultrasound systems based on a scanner grip are described. In some embodiments, an ultrasound system includes an ultrasound scanner having a touch sensitive surface and a processor that is configured to determine a grip orientation on the touch sensitive surface, and activate, based on the grip orientation, a region of the touch sensitive surface to accept a user input.

Typically, conventional ultrasound systems require that ultrasound operators divert their attention away from the patient and towards the ultrasound system, resulting in less than optimum patient care. Accordingly, systems, devices, and techniques are described herein for configuring an ultrasound system based on a scanner grip to avoid diverting of the operator's attention from the patient and to improve patient care comparing to conventional systems.

Embodiments described herein allow configuring and controlling an ultrasound system based on the operator's grip orientation of the scanner. In some embodiments, an ultrasound scanner body is touch sensitive (e.g., a touchscreen) or has touch sensitive areas. In some embodiments, an ultrasound system generates a grip map (e.g., location and pressure) indicative of the grip orientation. In some embodiments, one or more neural networks (NNs) processes the grip map, together with secondary inputs (e.g., the grip map may narrow to a class of solutions, but not a particular solution in the class). The ultrasound system can automatically configure and control the ultrasound machine based on an output of the one or more NNs (e.g., set an imaging parameter, examination type, etc.). In some embodiments, the ultrasound system generates, based on the grip map, an avatar/icon (e.g., of the scanner) for use in an AR/VR environment, as described in further detail below.

For example, the ultrasound system can determine a grip orientation of an ultrasound scanner, including, but not limited to, finger locations on the scanner, a palm location, whether the operator is left-handed or right-handed, etc. In some embodiments, the ultrasound system can determine, based at least in part on the grip orientation, a label, such as for an anatomy being imaged, an examination type, an imaging parameter, and the like. In some embodiments, the ultrasound system can then self-configure automatically and without user intervention based on the label, such as by setting the examination type for the ultrasound system. To determine the grip orientation, the scanner can include sensors (e.g., capacitive, pressure, resistive, or other sensors) for detecting the placement of a hand on the scanner, including finger locations, palm locations, fingerprints, etc.

Embodiments of the techniques described herein reduce the operator interaction with an ultrasound machine and are closer to a “plug and play” system than conventional ultrasound systems. In some embodiments, the touch sensitive region of the ultrasound scanner is dynamically changed to implement an adaptive user interface on the scanner, e.g., to locate, activate, and deactivate a button based on a finger position. In some embodiments, control of the AR/VR environment and/or ultrasound machine from the adaptive user interface on a scanner is provided. In some embodiments, an avatar for the AR/VR environment is generated from a grip map, as described in further detail below.

Reference in the specification to “one embodiment”, “an embodiment”, “one example”, or “an example” means that a particular feature, structure, or characteristic described in conjunction with the embodiment can be included in at least one embodiment. The appearances of the phrases “in one embodiment” or “in an embodiment” in various places in the specification do not necessarily all refer to the same embodiment. The processes depicted in the figures that follow are performed by processing logic that comprises hardware (e.g., circuitry, dedicated logic, etc.), software, firmware, or combinations thereof. Although the processes are described below in terms of some sequential operations, it should be appreciated that some of the operations described may be performed in a different order. Moreover, some operations may be performed in parallel rather than sequentially. Furthermore, it should be appreciated that not all operations of the processes described necessarily need to be performed.

In the specification, the term “and/or” describes three relationships between objects that may exist. For example, A and/or B may represent the following cases: only A exists, both A and B exist, and only B exist, where A and B may be singular or plural.

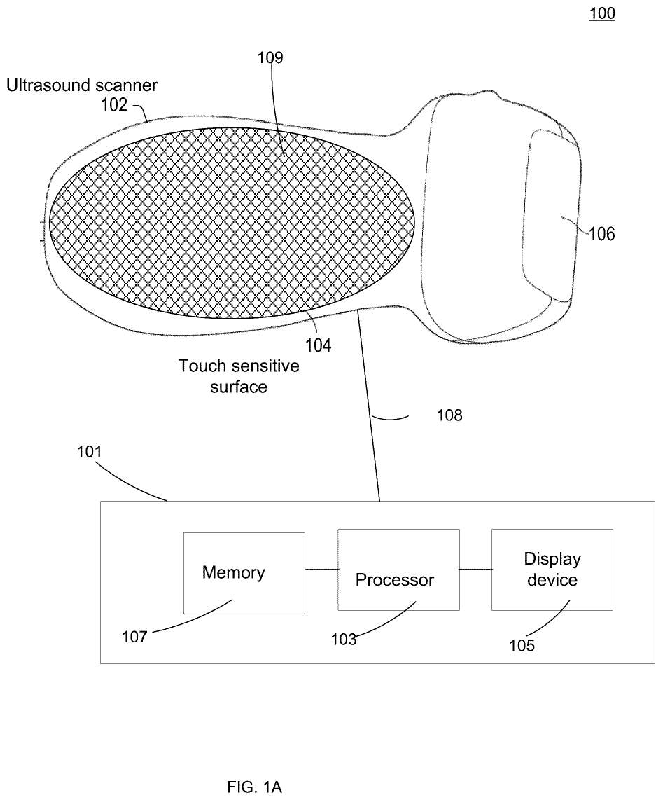

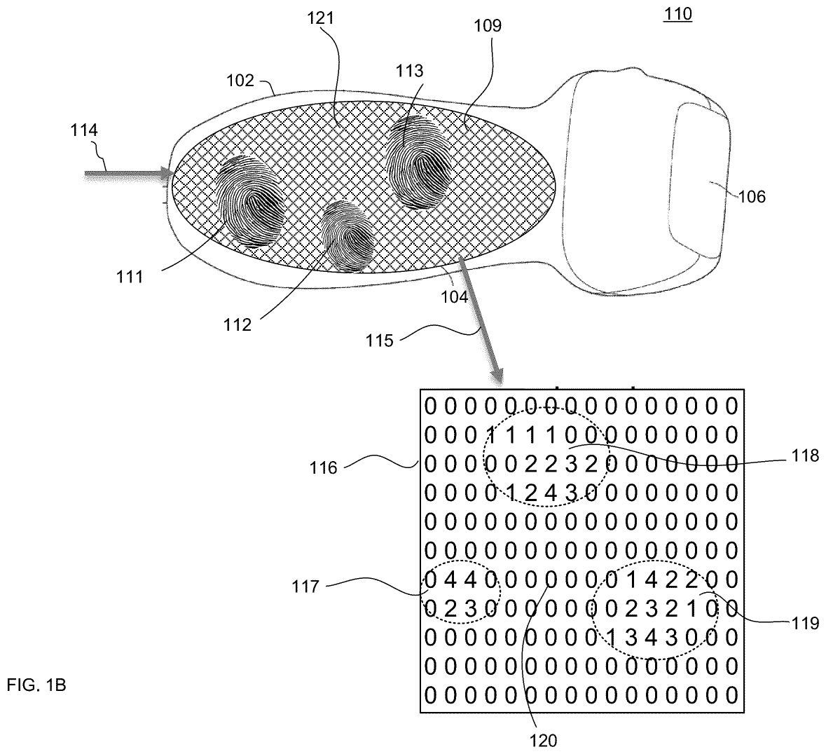

FIG. 1A is a view illustrating an ultrasound system 100 to detect a grip orientation according to some embodiments. As shown in FIG. 1A, an ultrasound system 100 includes an ultrasound scanner 102 that has a touch sensitive surface 104. The ultrasound scanner 102 is configured to generate ultrasound data based on reflections of ultrasound signals transmitted by the ultrasound scanner at an anatomy. In some embodiments, the ultrasound scanner 102 includes an ultrasound transducer array and an electronics coupled to the ultrasound transducer array to transmit ultrasound signals to a patient's anatomy and receive ultrasound signals reflected from the patient's anatomy. In some embodiments, the ultrasound scanner 102 is an ultrasound probe. In some embodiments, the ultrasound scanner 102 comprises an ultrasound patch having the touch sensitive surface. The patch can be placed on the skin of a patient. In some embodiments, the ultrasound scanner 102 comprises an ultrasound glove having the touch sensitive surface. The glove can be worn by a sonographer. The ultrasound system 100 includes a processor 103 that is configured to determine a grip orientation on the touch sensitive surface 104. The processor 103 can be implemented as part of a computing device 101, as illustrated in FIG. 1. Additionally or alternatively, the processor can be implemented as part of the scanner 102. In some embodiments, the touch sensitive surface 104 includes a pressure sensitive film (e.g., FUJIFILM's Prescale, a pressure measurement film) The processor 103 is configured to activate, based on the grip orientation, a region of the touch sensitive surface to accept a user input, e.g., the region can include one or more activated buttons that accept user inputs via touch. In some embodiments, the grip orientation includes at least one finger location, and the region of the touch sensitive surface to accept a user input is proximate to, and disjoint from, the at least one finger location. In some embodiments, the processor 103 is implemented to deactivate the region of the touch sensitive surface to accept the user input. In some embodiments, the processor 103 is implemented to deactivate, based on the grip orientation, an additional region of the touch sensitive surface to accept the user input.

As shown in FIG. 1A, the ultrasound system 100 includes a display device 105 coupled to processor 103 that is configured to generate and display an ultrasound image of the anatomy based on the ultrasound data generated by the ultrasound scanner 102. In some embodiments, the display device 105 is implemented to display a visual representation of the region of the touch sensitive surface 104. In some embodiments, the visual representation indicates a functionality for the user input, as described in further detail below. In some embodiments, the processor 103 is implemented to activate the region to accept the user input as a swiping/moving/change of grip gesture that controls at least one of a gain and a depth, and the ultrasound scanner 102 is implemented to generate ultrasound signals based on the at least one of the gain and the depth, as described in further detail below.

In some embodiments, the display device 105 is implemented to display an ultrasound image based on ultrasound data generated by the ultrasound scanner 102 and the processor 103 is implemented to adjust a zoom level of the ultrasound image based on a pressure of the user input on the region of the touch sensitive surface 104. In some embodiments, the zoom level of the ultrasound image is adjusted based on an amount of squeezing of the touch sensitive surface of the ultrasound scanner 102 by a user. In some embodiments, sliding a finger up/down on the touch sensitive surface of the scanner is used to adjust imaging gain/depth of the scanner. In some embodiments, one or more controls are adjusted in response to detection of a user squeezing harder on the touch sensitive surface with one finger, such as adjusting a zoom level based on the squeezing. In some embodiments, the ultrasound scanner 102 includes at least one light source (not shown in FIG. 1A) and the processor 103 is implemented to activate the at least one light source to emit light to indicate the region of the touch sensitive surface 104. In some embodiments, the processor 103 is configured to determine locations of pressure on the touch sensitive surface and amounts of that pressure at these locations, and determine, based on the locations and the amounts of the pressure and the ultrasound image, an elasticity of the anatomy. In some embodiments, the processor 103 is implemented to determine, based on the elasticity, a classification of the anatomy, such as, for example, a vein and/or an artery, as described in further detail below.

In some embodiments, the touch sensitive surface 104 is excluded from the surface of a lens 106 of the ultrasound scanner, and the processor 103 is implemented to determine the amount of pressure in a direction towards the surface of the lens 106. In some embodiments, the touch sensitive surface 104 includes the surface of the lens 106 of the ultrasound scanner, and at least some of the locations of pressure are on the surface of the lens 106. In some embodiments, the processor 103 is implemented to determine that the amount of pressure corresponds to an excessive pressure, and the display device 105 is implemented to display a warning that indicates the excessive pressure.

In some embodiments, the processor 103 is implemented to determine, based on at least one of the locations and the amount of pressure, an amount of coupling/uncoupling of the ultrasound scanner and a patient. The processor 103 can then adjust, based on the amount of coupling/uncoupling, at least one imaging parameter, as described in further detail below. In some embodiments, the touch sensitive surface 104 includes a pressure sensitive material deposited on the lens 106. The processor 103 can determine, based on at least one of the locations and the amount of pressure on the pressure sensitive material when the scanner (e.g., an ultrasound probe) is decoupled from the patient (e.g., when a user inadvertently lifts part of the probe). Hence, the ultrasound system 100 can condition the grip map based on an amount of coupling between the probe and a patient, such as to feed the ultrasound imaging data into an image quality score, and/or to exclude from NN processing the ultrasound imaging data of uncoupled from scanner spots of a patient's anatomy. In this way, the power of the ultrasound system can be saved, ultrasound imaging frame rate of the coupled spots of the patient anatomy can be increased, and/or the ultrasound beam can be re-programmed to use only the contacted part of the probe, when the probe is lifted partially from patient. In some embodiments, a grip map generated by the ultrasound system can be conditioned based on the amount of coupling/uncoupling between the probe and a patient. In an example, an amount of coupling of the scanner to a patient, such as a percentage, indicator of well-coupled and/or poorly coupled regions, etc., is provided to the NN as a secondary (e.g., conditional) input, in addition to a grip map.

In some embodiments, the processor 103 is configured to set, based on the grip orientation, an imaging parameter for at least one of the display device 105 and the ultrasound scanner 102. In some embodiments, the processor 103 is implemented to set the imaging parameter to control beamforming of at least one of the ultrasound signals and the reflections. In some embodiments, the processor 103 is implemented to determine, based on the grip orientation, a patient anatomy, and set the imaging parameter based on the patient anatomy. In some embodiments, the imaging parameter includes at least one of a depth and a gain for the ultrasound signals transmitted by the ultrasound scanner 102. In some embodiments, the ultrasound scanner 102 includes an inertial measurement unit (not shown in FIG. 1A) configured to generate orientation data, and the processor 103 is implemented to determine, based on the orientation data, an orientation of the ultrasound scanner. In some embodiments, the ultrasound system 100 includes a neural network (not shown in FIG. 1A) implemented to determine the imaging parameter based on the grip orientation and at least one of the ultrasound image, the orientation of the ultrasound scanner, an operator identification, and a voice command, as described in further detail below.

As shown in FIG. 1A, computing device 101 is coupled to the ultrasound scanner 102 via a communication link 108. In some embodiments, communication link 108 is a wireless communication link. In some embodiments, communication link 108 is a wired communication link. As shown in FIG. 1A, computing device 101 includes a memory 107 coupled to the processor 103 and display device 105 that is configured to display an ultrasound image. In some embodiments, computing device 101 is a tablet, a smart phone, an ultrasound machine, a heads-up display, smart glasses/goggles, or other computing device. In one example, at least part of the computing device 101 is included as part of the ultrasound scanner 102, such as the memory 107 and/or the processor 103.

As shown in FIG. 1A, touch sensitive surface 104 includes a sensor region 109 including one or more sensors to detect a grip orientation. In some embodiments, the one or more sensors of the sensor region 109 are under the touch sensitive surface 104. In some embodiments, the one or more sensors of the sensor region 109 are on the touch sensitive surface 104. In some embodiments, the one or more sensors of the sensor region 109 are capacitive sensors that measure a capacitance, or change in capacitance, caused by a user's touch or proximity of touch, as is common in touchscreen technologies. Additionally or alternatively, the one or more sensors of the sensor region 109 are pressure sensors configured to determine an amount of pressure caused by the user's grip on the scanner. In some embodiments, the amount of pressure determined by the one or more sensors is indicative of the amount of coupling/uncoupling between the ultrasound scanner and a patient, as described in further detail below.

In FIG. 1A, the touch sensitive surface 104 is shown for clarity as an ellipsoid. However, the touch sensitive surface 104 of the ultrasound scanner 102 can be of any suitable shape. In some embodiments, the touch sensitive surface 104 substantially covers the surface of the scanner, e.g., the touch sensitive surface can cover all of the ultrasound scanner 102 except the lens 106. In some embodiments, the touch sensitive surface 109 substantially covers of the ultrasound scanner 102 including the lens 106.

FIG. 1B is a view illustrating an ultrasound system 110 that generates 115 one or more grip maps to detect a grip orientation according to some embodiments. In some embodiments, the ultrasound system 110 depicted in FIG. 1B represents one of the ultrasound systems described in the present disclosure, e.g., ultrasound system 100. As shown in FIG. 1B, the ultrasound system 110 includes an ultrasound scanner 102 having a touch sensitive surface 104. The touch sensitive surface 104 includes a sensor region 109 including one or more sensors, as described above. The ultrasound system can use data 114 captured by the one or more sensors of the sensor region 109 to configure an ultrasound machine (not shown in FIG. 1B) coupled to the ultrasound scanner 102. In some embodiments, the ultrasound system receives the sensor data 114 from sensors of the sensor region 104 and generates a data structure 116 indicative of the grip orientation based on the sensor data. In some embodiments, the data structure 116 is an array including one or more columns, and/or one or more rows of the sensor data. In some embodiments, the data structure 116 is a grip map. In some embodiments, the data structure 116 is a two-dimensional grid (e.g., a matrix).

In some embodiments, a node of the grid, such as a node 120, represents a sensor of the sensor region 109, and include the location and/or pressure data from that sensor. In some embodiments, each intersection of the cross hatching, such as an intersection 121 in the sensor region 109, corresponds to a sensor for determining the grip orientation, and hence a node in the two-dimensional grid. In some embodiments, the sensor data include a binary indicator that indicates the presence or absence of a user hand on or proximate to the sensor. For example, a “1” for a sensor can indicate that the user's hand is in a grip orientation that covers the sensor, and a “0” for the sensor can indicate that the user's hand is in a grip orientation that does not cover the sensor. Additionally or alternatively, the sensor data can include a multi-level indicator that indicates an amount of pressure on the sensor, such as, for example, an integer scale from zero to four. For example, a “0” can indicate that no pressure from the user's hand is detected at the sensor, and a “1” can indicate a small amount of pressure from the user's hand is detected at the sensor. A “2” can indicate a larger amount of pressure from the user's hand is detected at the sensor than a “1”, and a “4” can indicate a maximum amount of pressure from the user's hand is detected at the sensor, as shown in FIG. 1B. In some embodiments, detecting the grip orientation includes determining one or more finger locations, such as a finger location 111, a finger location 112 and a finger location 113 on the touch sensitive surface 104 based on sensor data, e.g., a cluster of sensor data 117, a cluster of sensor data 118, and a cluster of sensor data 119 respectively, as described in further detail below. In some embodiments, the grip map indicating one or more finger locations is displayed on a display to show a correspondence between the finger locations on the scanner and a control/function to a user. In other words, a grip map is displayed on a display to indicate to the user where the controls on the scanner are located relative to the finger locations, or what finger controls what function. The display can also display an identifier of the user controls/functions, such as a label or icon for “gain”, another label or icon for “depth”, etc., proximate to the finger locations. In an example, a user can select what function is controlled by which finger, such as by assigning via a user interface of the display a gain function to an index finger and a depth function to a middle finger.

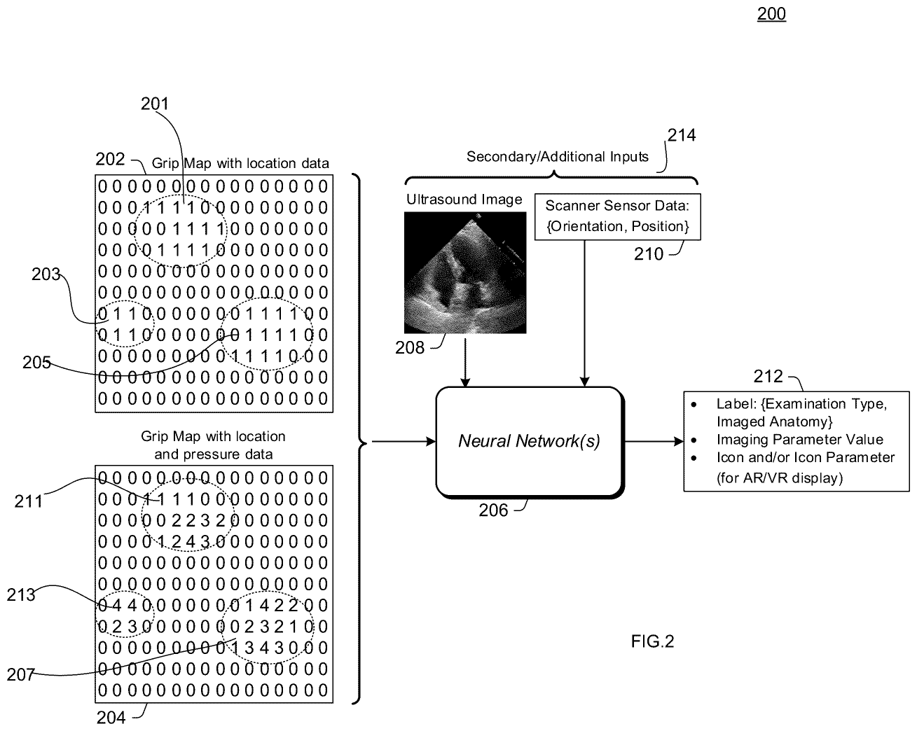

FIG. 2 is a view 200 illustrating an example of use of grip maps that represent a grip orientation according to some embodiments. A grip map 202 is an example of a grip map that includes location data, and thus includes an array of binary values. Here, a “1” indicates the presence of contact of, or proximity to, a sensor of the ultrasound scanner 102, and a “0” indicates the absence of contact/proximity for the sensor. In some embodiments, grip map 204 is an example of a grip map that includes location and pressure data in the scale of 0 to 4. In each of the grip map 202 and the grip map 204, three clusters of sensor location/pressure data, such as clusters 201, 203, 205, 207, 211 and 213 are illustrated and encircled by ellipses for clarity. In some embodiments, clusters of sensor location data 201, 203, 205 indicate finger locations on the ultrasound scanner. In some embodiments, clusters of sensor location and pressure data 207, 211, 213 indicate finger locations and an amount of pressure for each of the finger locations on the ultrasound scanner.

One or more grip maps 202 and 204 are provided as input to one or more neural networks 206, as shown in FIG. 2. In some embodiments, the ultrasound system selects a neural network (NN) to process the grip map based on any suitable factor, such as a user-selection, an output generated by another neural network, an ultrasound image, and the like. In some embodiments, a plurality of NNs operate in series to process the sensor data based on a confidence level for a NN inference. For example, a first neural network is selected to process a grip map. The first neural network can generate an inference (e.g., a label, and imaging parameter value, an icon and/or icon parameter (for augmented reality (AR)/virtual reality (VR) display) and a confidence level for the inference. If the confidence level for the inference is below a threshold confidence level (e.g., less than a 66% confidence, with 100% representing total confidence and 0% representing no confidence), then the ultrasound system can disable the first neural network and select a second neural network as the neural network 206 to process the grip map. In some embodiments, a plurality of NNs operate simultaneously to process the sensor data. In some embodiments, one or more neural networks 206 includes a first NN that is configured to process an ultrasound image and generate a feature map and a second NN that inputs the feature map generated by the first NN and one or more secondary inputs 214 and generates an output 212. The neural network 206 can be provided any suitable secondary (or additional) inputs. In some embodiments, the grip map alone may not be sufficient to determine an appropriate examination type, imaging parameter, etc., but when combined with one or more secondary inputs 214 can include sufficient information to determine an appropriate examination type, imaging parameter, etc. In other words, the information in the grip map can correspond to a subset of examination types, imaging parameters, etc., but may not be unique to a particular examination type or imaging parameter in the subset.

In one example, the ultrasound system provides an ultrasound image 208 as one or more secondary inputs 214 to the neural network 206. The ultrasound image 208 can be generated by the ultrasound system based on ultrasound data captured by the ultrasound scanner 102. In some embodiments, one or more secondary inputs 214 include scanner sensor data 210 indicating, e.g., a grip/scanner orientation, a grip/scanner position, an amount of coupling/uncoupling between the probe and a patient. For instance, the ultrasound scanner 102 can include one or more location and/or orientation sensors that are configured to generate location and/or orientation data for the ultrasound scanner 102. As an example, the ultrasound scanner 102 can include an inertial measurement unit (IMU) that can measure one or more of force, acceleration, angular rate, and magnetic field. An IMU can include a combination of accelerometers, gyroscopes, and magnetometers, and generate location and/or orientation data including data representing six degrees of freedom (6DOF), such as yaw, pitch, and roll angles in a coordinate system. Typically, 6DOF refers to the freedom of movement of a body in three-dimensional space. For example, the body is free to change position as forward/backward (surge), up/down (heave), left/right (sway) translation in three perpendicular axes, combined with changes in orientation through rotation about three perpendicular axes, often termed yaw (normal axis), pitch (transverse axis), and roll (longitudinal axis). Additionally or alternatively, the ultrasound system can include a camera to determine location and/or orientation data for the ultrasound scanner 102.

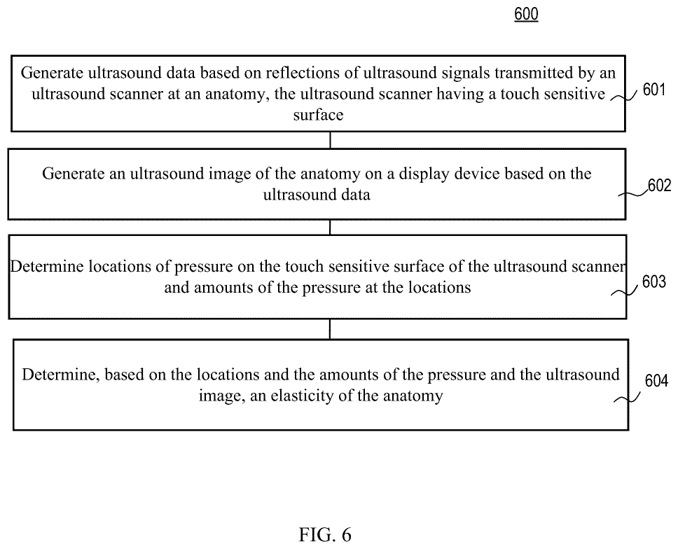

In some embodiments, one or more secondary inputs 214 that the ultrasound system can provide to the neural network 206 include the value of an imaging parameter (e.g., a gain or depth), a probe identifier (e.g., an indicator of probe type, such as linear, curved, phased array, etc.), an initialization coordinate in space (e.g., a starting position of the scanner on a patient), metadata (e.g., identifying a user for the system to learn and predict the user actions), sensor data voice (e.g., spoken by a sonographer and/or patient), gaze tracking data, patient orientation/position, image of the patient, or other secondary input data. In some embodiments, the ultrasound system listens to voice as a secondary input only when a predetermined condition is met, for example, when a sonographer squeezes the scanner. In some embodiments, the ultrasound system provides an image segmentation based on where a user is gazing. In some embodiments, a user interface (UI) control is mapped to a location on the scanner and the ultrasound system selects the UI control based on a location of a user's gaze. In some embodiments, the system determines that a user looks at a part of an image on the screen, and then manipulates the grip, e.g., the system determines that the grip control is for the part of image on the screen at which the user looks. In some embodiments, the sensor data represent the pressure data on a transducer face and/or side of the scanner, smearing/movement/change of the grip map data to infer the downward pressure to a patient, or grip map/pressure data to perform an image correction, as described in further detail below with respect to FIG. 6.

The neural network 206 can combine the grip map input with the secondary input in any suitable way. In one example, the neural network 206 concatenates the secondary input and the grip map, and processes the concatenated data at the top (first) layer of the neural network 206. Additionally or alternatively, the neural network 206 can process the grip map with one or more layers of the neural network and concatenate the results with the secondary input for subsequent layers of the neural network. Additionally or alternatively, the neural network 206 can process the grip map with a first section of the neural network and the secondary input with a second section of the neural network. The neural network 206 can combine one or more of the results of the first and second sections with one or more of the grip map and the secondary input.

Based on the grip map and the secondary input, the neural network 206 can generate an output 212. In some embodiments, the output 212 includes a label. Examples of a label include an examination type (e.g., cardiac, respiratory, etc.), an examination protocol (e.g., eFAST, FAST, BLUE, FATE, FALLS, etc.), an imaged anatomy (e.g., bladder), and the like. Additionally or alternatively, the neural network 206 can generate a value of an imaging parameter, such as a depth or gain setting. In some embodiments, the ultrasound system can automatically and without user intervention configure at least one of the ultrasound scanner 102 and a computing device coupled to the ultrasound scanner 102, such as an ultrasound machine or tablet, based on a label or imaging parameter generated by the neural network 206. In this way, the operator of the ultrasound system does not need to divert their attention from the patient to configure the ultrasound system, unlike the operator of a conventional ultrasound system.

In some embodiments, the neural network 206 generates an icon for insertion into an AR/VR environment. For example, the neural network 206 can generate an icon of the ultrasound scanner 102 that can be inserted into an AR/VR environment. Additionally or alternatively, the neural network 206 can generate an icon parameter for insertion into the AR/VR environment, such as, for example, an orientation or positioning of the icon within the AR/VR environment. Additionally or alternatively, the icon parameter can determine a point of view within the AR/VR environment, such as a point of view according to the ultrasound scanner 102 or according to an operator who is holding the ultrasound scanner. The AR/VR environment can include an ultrasound image overlaid with an icon generated by the neural network 206.

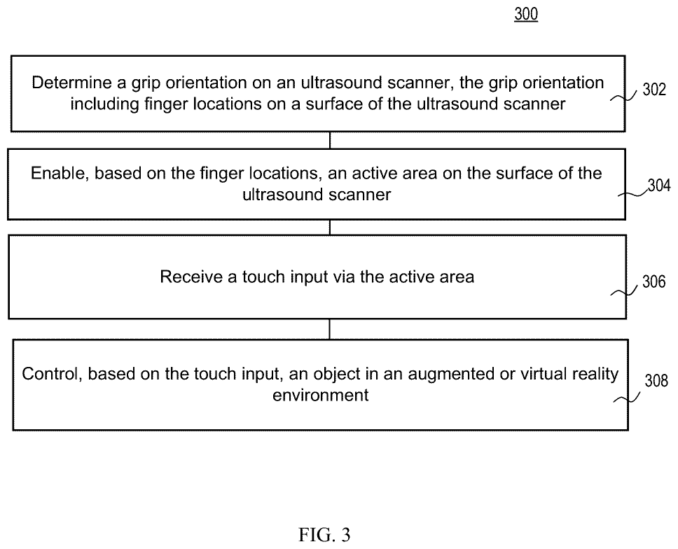

In some embodiments, the ultrasound system configures a region of the ultrasound scanner 102 to accept a user input, such as by enabling one or more buttons in the sensor region 109. The operator can control an object in the AR/VR environment via the buttons, such as an icon of the scanner, an avatar of the operator, etc., as discussed below with respect to the method illustrated in FIG. 3.

FIG. 3 illustrates a method 300 implemented by a computing device (e.g., an ultrasound machine, tablet, ultrasound scanner, combinations thereof, etc.) for controlling an object in an AR/VR environment based on a scanner grip orientation according to some embodiments. In some embodiments, method 300 is performed by processing logic that may comprise hardware (circuitry, dedicated logic, etc.), software (such as is run on a general-purpose computer system or a dedicated machine), firmware, or combinations thereof. Referring to FIG. 3, method 300 determines a grip orientation on an ultrasound scanner (block 302), e.g., using sensors on the ultrasound scanner, such as capacitive and/or pressure sensors. The grip orientation can include finger locations on a surface of the ultrasound scanner. Based on the finger locations, the ultrasound system enables an active area on the surface of the ultrasound scanner (block 304). In some embodiments, the active area on the surface of the ultrasound scanner is displayed as one or more controls, e.g., virtual buttons, icons, or other controls to receive a user's input. In some embodiments, the one or more virtual controls are displayed based on the grip orientation and/or an amount of pressure applied to the touch sensitive surface of the scanner. For example, one control can be displayed for one grip orientation and/or the amount of pressure, and another control can be displayed for another grip orientation and/or amount of pressure. In an example, prior to the active area being enabled at block 304, the one or more controls (e.g., buttons), are configured to not accept user input. For instance, the controls are disabled.

In some embodiments, the active area excludes the finger locations, so that a user can move a finger from the grip orientation to the active area to apply an input to a control (e.g., a button) activated in the active area. Method 300 also includes receiving a touch input via the active area (block 306) and having the ultrasound system control, based on the touch input, an object in an augmented or virtual reality environment (block 308). In some embodiments, the object in the augmented or virtual reality environment represents the ultrasound scanner. In some embodiments, method 300 can be used for virtual training, e.g. using the scanner to press on an anatomy in a VR space and see the effecting VR space without actually imaging people and/or for telemedicine. Additionally or alternatively to controlling an object in an AR/VR environment, the ultrasound system can set an imaging parameter based on the touch input, such as by setting a gain, depth, examination preset, beamformer configuration, and the like.

In some embodiments, the ultrasound system determines the grip orientation as a left-handed grip or a right-handed grip, and determines a location on the surface of the ultrasound scanner for the active area based on the determination of the left-handed grip or the right-handed grip. Additionally or alternatively, the ultrasound system can determine one location of the finger locations as corresponding to an index finger, and determine a location on the surface of the ultrasound scanner for the active area based on the one location, so that the user can easily move the index finger to the active area. In an embodiment, the ultrasound system can determine at least one fingerprint corresponding to one or more of the finger locations, and authenticate, based on the at least one fingerprint, a user of the ultrasound scanner.

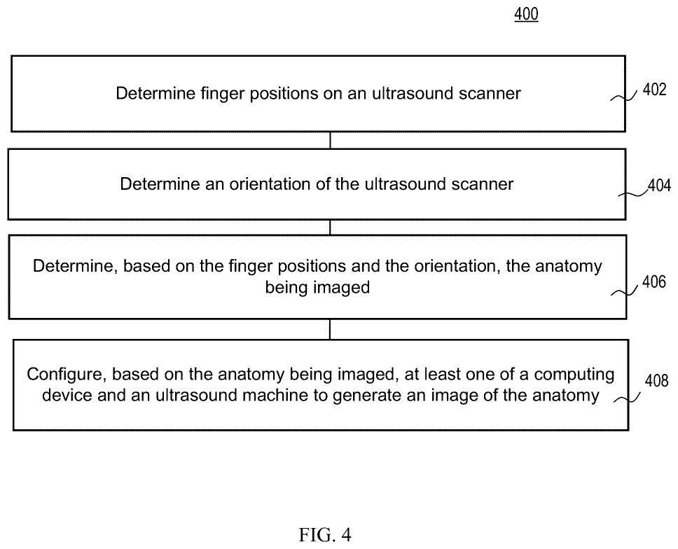

FIG. 4 illustrates a method 400 for configuring a device (e.g., an ultrasound machine or a computing device coupled to a scanner, such as a tablet) to image an anatomy based on a scanner grip orientation according to some embodiments. The method can be implemented by any suitable computing device, including an ultrasound machine, a computing device coupled to a scanner, the scanner itself, or combinations thereof. In some embodiments, method 400 is performed by processing logic that may comprise hardware (circuitry, dedicated logic, etc.), software (such as is run on a general-purpose computer system or a dedicated machine), firmware, or combinations thereof.

Referring to FIG. 4, method 400 determines finger positions on an ultrasound scanner (block 402) and an orientation of the ultrasound scanner (block 404). In some embodiments, a computing device determines the finger positions including generating a grip map, as discussed above. In some embodiments, the computing device receives capacitive data from capacitive sensors coupled to the ultrasound scanner and determines the finger positions based on the capacitive data. In some embodiments, the ultrasound scanner includes sensors, such as, for example, an IMU, to determine the orientation of the ultrasound scanner. In one example, determining the orientation of the ultrasound scanner includes determining a position of the ultrasound scanner relative to a marker, such as, for example, a marker in the examination room and/or a marker on the patient (e.g., a patient marker). In some embodiments, the ultrasound system receives at least one of pitch, yaw, and roll data from the ultrasound scanner and determines the orientation of the ultrasound scanner based on at least one of pitch, yaw, and roll data. Additionally or alternatively, determining the orientation of the ultrasound scanner can be based on the finger positions, such as by comparing the finger positions to a database of finger positions for right-handed and left-handed grips when the scanner is held in different orientations In one example, receiving at least one of pitch, yaw, and roll data from the ultrasound scanner includes receiving no more than two of the pitch, yaw, and roll data.

In some embodiments, method 400 also determines an anatomy being imaged based on the finger positions and the orientation (block 406). For example, a neural network can process the grip map representing the finger positions and a vector of coordinates that represent the scanner orientation to determine the anatomy being imaged. In an example, the ultrasound system determines a grip of the ultrasound scanner as a left-handed grip or a right-handed grip, and determining the anatomy being imaged is based on the grip. Based on the anatomy being imaged, at least one of the computing device and an ultrasound machine is configured to generate an image of the anatomy (block 408). Configuring the computing device or the ultrasound machine can include setting at least one of a gain, a depth, and an examination type.

In some embodiments, the computing device receives pressure data from a pressure sensor coupled to the ultrasound scanner. Determining the anatomy being imaged can be based on the pressure data. For example, the grip map can include the pressure data, and a neural network can process the grip map to determine the anatomy being imaged. Additionally or alternatively, the computing device can determine a patient orientation, such as whether they are laying on their back, side or stomach, and determining the anatomy being imaged can be based on the patient orientation. For example, the patient orientation can be assigned a number (such as “1” representing the patient laying on their back, “2” representing the patient laying on their stomach, “3” representing the patient laying on their left side, “4” representing the patient laying on their right side, and the like), and the number can be input to the neural network as a secondary, or additional input. In some embodiments, the ultrasound system includes a camera configured to capture an image of the patient and the patient orientation, and this image is provided to the neural network as a secondary, or additional input.

In some embodiments, the ultrasound system selects, based on the anatomy being imaged, a neural network from a plurality of available neural networks. For example, the computing device and/or ultrasound machine can include a plurality of available neural networks to implement, such as one neural network that has been trained for cardiac imaging, another neural network that has been trained for bladder scans, etc. The computing device and/or ultrasound machine can receive ultrasound data from the ultrasound scanner, and enable, automatically and without user intervention, the neural network to generate, based on the ultrasound data, an inference for the anatomy. The inference can include at least one of a blood vessel classification, a cardiac ejection fraction, a determination of free fluid, and a pathology identification.

In some embodiments, the ultrasound system configures, based on the finger positions, the ultrasound scanner to accept at least one user input. For example, configuring the ultrasound scanner can include enabling an area of the ultrasound scanner as a button to accept at least one user input. For example, the ultrasound system can enable the area adjacent to a finger location as a button to accept user input, so that the operator does not need to remove their hand from the scanner to activate the button, but rather just move their finger a small amount to reach the button. Moreover, the ultrasound system can be configured to disable the area of the ultrasound scanner as the button to accept the at least one user input. For instance, if the user changes their grip on the scanner, such as changing from right hand to left hand, the ultrasound system can disable the area/button. Additionally or alternatively, the ultrasound system can configure, based on the finger positions, a surface region of the ultrasound scanner to reject user inputs, e.g., by disabling a button on the surface region. In some embodiments, determining the finger positions includes determining at least one fingerprint. For example, the ultrasound system can include a fingerprint reader that recognizes fingerprints from the sensor data (e.g., capacitive data) from the ultrasound scanner. Based on the fingerprint, the ultrasound system can execute a user authentication to verify an operator of the ultrasound system and permit its use by the operator. In some embodiments, the touch sensitive surface of the ultrasound scanner includes a light emitting diode (LED) fabric (e.g., a flexible organic LED screen) under a shell that illuminates locations of buttons on the scanner (e.g., underneath and around the buttons). For instance, the light can trace a perimeter of an activated button, and the light can be disabled when the button is disabled.

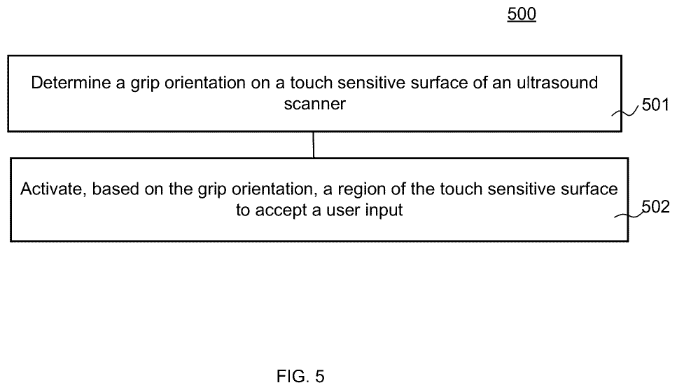

FIG. 5 illustrates a method 500 to configure an ultrasound system based on a scanner grip according to some embodiments. The method can be implemented by any suitable computing device, including an ultrasound machine, a computing device coupled to a scanner, the scanner itself, or combinations thereof. In some embodiments, method is performed by processing logic that may comprise hardware (circuitry, dedicated logic, etc.), software (such as is run on a general-purpose computer system or a dedicated machine), firmware, or combinations thereof. Referring to FIG. 5, method 500 includes determining a grip orientation on a touch sensitive surface of an ultrasound scanner at block 501 and activating a region of the touch sensitive surface to accept a user input based on the grip orientation at block 502, as described above.

In some embodiments, the grip orientation includes at least one finger location, and the region is proximate to, and disjoint from, the at least one finger location. In some embodiments, the method 500 includes deactivating the region of the touch sensitive surface to accept the user input, such as when the operator moves their hand. In some embodiments, the method 500 includes deactivating, based on the grip orientation, an additional region of the touch sensitive surface to accept the user input. In some embodiments, the method 500 includes displaying a visual representation of the region of the touch sensitive surface on a display device. The visual representation can indicate a functionality for the user input. In some embodiments, the method 500 includes activating the region of the touch sensitive surface to accept the user input as a swiping gesture that controls at least one of a gain and a depth. In some embodiments, the method 500 includes generating ultrasound signals based on at least one of the gain and the depth. In some embodiments, the method 500 includes displaying, on a display device, an ultrasound image based on ultrasound data generated by the ultrasound scanner. In some embodiments, the method 500 includes adjusting a zoom level of the ultrasound image based on a pressure of the user input on the region of the touch sensitive surface. For instance, the zoom level of the ultrasound image can be adjusted based on an amount of squeezing of the touch sensitive surface of the ultrasound scanner by a user. Squeezing harder can increase the zoom level, an reducing the pressure of the squeezing can decrease the zoom level. A double squeeze can freeze the zoom level.

In some embodiments, sliding a finger up/down on the touch sensitive surface of the scanner is used to adjust imaging gain/depth of the scanner. In some embodiments, one or more virtual controls are adjusted in response to detection of a user pressing (e.g., as part of squeezing) harder on the touch sensitive surface with one finger. In some embodiments, the display device displays a visual representation of the activated buttons and the finger locations to help a user orient their hand to the controls. For example, a visual representation of the controls can be configurable e.g. based on a user ID that is associated with one or more user preferences (e.g., a finger, a left hand or a right hand used to operate a control). For example, a sonographer may prefer to use their pinky finger to control gain, and a visual representation of the control to control the gain is adjusted such that the sonographer can use their pinky finger to control the gain. In some embodiments, the method 500 includes displaying a user identification (ID) on a display device based on the grip map. For example, the ultrasound system can determine the user ID from the grip map, such as by comparing the grip map to a database of grip maps associated with different users, and then display the determined user ID on the display device. In some embodiments, the ultrasound scanner includes at least one light source and the method 500 includes activating the at least one light source to emit light to indicate the region of the touch sensitive surface. For instance, the light can illuminate an area, perimeter, etc., of the region.

FIG. 6 illustrates a method 600 to determine an elasticity of an anatomy according to some embodiments. The method can be implemented by any suitable computing device, including an ultrasound machine, a computing device coupled to a scanner, the scanner itself, or combinations thereof. In some embodiments, the method is performed by processing logic that may comprise hardware (circuitry, dedicated logic, etc.), software (such as is run on a general-purpose computer system or a dedicated machine), firmware, or combinations thereof. Referring to FIG. 6, method 600 includes generating ultrasound data based on reflections of ultrasound signals transmitted by an ultrasound scanner at an anatomy at block 601. The ultrasound scanner has a touch sensitive surface. At block 602, method 600 includes generating an ultrasound image of the anatomy on a display device based on the ultrasound data. Method 600 also includes determining locations of pressure on the touch sensitive surface of the ultrasound scanner and the amount of the pressure at those locations (block 603), and based on the locations, the amount of pressure, and the ultrasound image, determines an elasticity of the anatomy (block 604). In some embodiments, the method 600 includes determining, based on the elasticity, a classification of the anatomy as at least one of a vein and an artery.

In some embodiments, the touch sensitive surface is excluded from a lens surface of the ultrasound scanner. In some embodiments, the method 600 includes to determine the amounts of the pressure in a direction towards the lens surface. In some embodiments, the touch sensitive surface includes a lens surface of the ultrasound scanner. In some embodiments, at least some of the locations of the pressure are on the lens surface. In some embodiments, the method 600 includes determining that the amounts of the pressure correspond to an excessive pressure. In some embodiments, the method 600 includes displaying a warning that indicates the excessive pressure on a display device. In some embodiments, the method 600 includes determining, based on at least one of the locations and the amounts of the pressure, an amount of uncoupling of the ultrasound scanner from a patient, and adjusting, based on the amount of uncoupling, at least one imaging parameter.

In some embodiments, the elasticity of the anatomy is determined based on real time image and the pressure data in order to determine if a vein is compressing, to measure tissue firmness, and/or to measure tissue density. In some embodiments, the neural network 206 determines and outputs the elasticity of the anatomy based on real time image and the pressure data. In some embodiments, the grip map/pressure data are used to correct an ultrasound image generated by the system. In some embodiments, the pressure data are used to determine whether the anatomy is vein versus an artery. For example, the pressure data (e.g., downward pressure on a patient) can be used in ultrasound-guided peripheral IV (PIV) to determine if a blood vessel is a vein or an artery. For example, the ultrasound system can determine that the blood vessel is more likely an artery than a vein when the pressure data indicating a downward pressure on a blood vessel exceeds a predetermined threshold, and the blood vessel does not collapse. For example, the pressure data (e.g., downward pressure on a patient) can be used to avoid damaging the patient during a medical procedure and/or examination. In some embodiments, the system provides a haptic feedback to a user based on the pressure data, for example, to indicate that a user is pressing onto a patient anatomy too hard. Such feedback may be used to prevent injury of the patient and or the user and help prevent carpal tunnel syndrome.

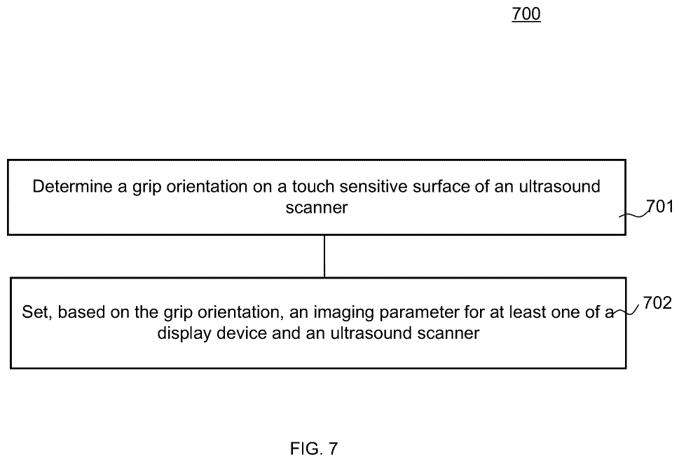

FIG. 7 illustrates a method 700 to set an imaging parameter for an ultrasound system according to some embodiments. The method can be implemented by any suitable computing device, including an ultrasound machine, a computing device coupled to a scanner, the scanner itself, or combinations thereof. In some embodiments, the method is performed by processing logic that may comprise hardware (circuitry, dedicated logic, etc.), software (such as is run on a general-purpose computer system or a dedicated machine), firmware, or combinations thereof. Referring to FIG. 7, method 700 includes determining a grip orientation on a touch sensitive surface of an ultrasound scanner (block 701) and setting an imaging parameter for at least one of a display device and an ultrasound scanner based on the grip orientation (block 702), as described above. In some embodiments, method 700 includes setting the imaging parameter to control beamforming of at least one of the ultrasound signals and the reflections. In some embodiments, method 700 includes determining, based on the grip orientation, a patient anatomy, and set the imaging parameter based on the patient anatomy. For instance, the ultrasound system can set the gain to one value for an anatomy corresponding to a lung, and to a second value for an anatomy corresponding to a blood vessel. In some embodiments, the imaging parameter includes at least one of a depth and a gain for the ultrasound signals transmitted by the ultrasound scanner. In some embodiments, the ultrasound scanner includes an inertial measurement unit. The method 700 can include generating, by the inertial measurement unit, orientation data, and determining, based on the orientation data, an orientation of the ultrasound scanner. In some embodiments, the ultrasound system includes a neural network. The method 700 can include determining, by the neural network, the imaging parameter based on the grip orientation and at least one of the ultrasound image, the orientation of the ultrasound scanner, an operator identification, and a voice command.

In some embodiments, the ultrasound system learns (e.g., adapts, updates) based on a grip orientation of the ultrasound scanner and current actions to predict next actions. In some embodiments, the ultrasound system provides feedback and/or guidance to an operator based on what the system predicts the user is trying to do. This can improve training/education and help the user to be successful. In some embodiments, a unique grip position (map) on an ultrasound scanner is provided as a security measure to log into the scanner. For example, a user can place their finger on the touch sensitive surface of the ultrasound scanner and the scanner can authenticate the user (e.g., confirm their identity, access level, job title, combinations thereof, extract a user ID, and the like) based on their fingerprint. In some embodiments, the grip map generated by the ultrasound system is correlated with accelerometer data, to determine where in an examination protocol (e.g., eFAST) a user is. In some embodiments, a section of an anatomy to be scanned is determined based on the grip orientation on the touch sensitive surface of the ultrasound scanner. For example, one grip orientation on the touch sensitive surface of the ultrasound scanner can indicate a lung scan is being performed while another orientation on the touch sensitive surface of the ultrasound scanner can indicate a heart scan is being performed.

In some embodiments, the grip map is used by the ultrasound system to identify and discourage bad ways to grip the scanner, that can result in impairment to the operator, such as carpal tunnel syndrome In some embodiments, the one or more grip maps generated by the ultrasound system are used to improve ultrasound scanner design. In some embodiments, meta material is used to reconfigure a shape of an ultrasound probe. For example, the shape of the ultrasound probe can be reconfigurable, via the meta material of the probe, based on the data collected from the one or more grip maps. For example, the meta material of the probe can reconfigure itself based on where a user's hands are on the probe. For instance, using pressure data, the system can determine a shape of the probe for a scan, such as to better fit a user based on the user's grip map. As an example, the probe can be reconfigured to emphasize a ridge on the probe for a user's hand to rest on for better control of the probe. In some embodiments, the system scores the shape of the probe based on the quality of the image. The score can be stored in a database and used to determine a future shape of the probe. In some embodiments, the system reconfigures the shape of the probe to fit a user based on a user ID and/or history (including a previous shape of the probe for a user, and the score for the shape of the probe when used by the user).

In some embodiments, a voice control (e.g., “set index finger as gain”), a user's gaze tracking, or other biological data (e.g., breathing cycle) of a user is used to enable a user interface on the touch sensitive surface on the scanner. In some embodiments, an ultrasound scanner reports about what environment it is in based on the grip map, e.g., on a flat surface, gel, or wrapped in a blanket. In some embodiments, the ultrasound system can detect and report, based on one or more grip map and accelerometer data, if the scanner is being stolen. In some embodiments, the ultrasound system includes a sleeve that extends over the ultrasound probe. In some embodiments, the sleeve is reconfigurable based on the grip map to protect from dropping of the probe and/or to provide better cleaning. In some embodiments, the ultrasound system uses the one or more grip maps for safe login to the scanner. In some embodiments, the ultrasound system displays one or more grip maps (e.g., finger locations) on a display device to show to a user which finger is assigned to which control/function. In some embodiments, the display device has an LED fabric under the shell that illuminates where control buttons are on the surface (underneath and around the finger locations). In some embodiments, the display device includes a flexible organic light-emitting diode (OLED) screen. In some embodiments, the ultrasound system correlates the grip map with accelerometer data, to determine which portion of an exam (e.g., eFAST) is currently performed.

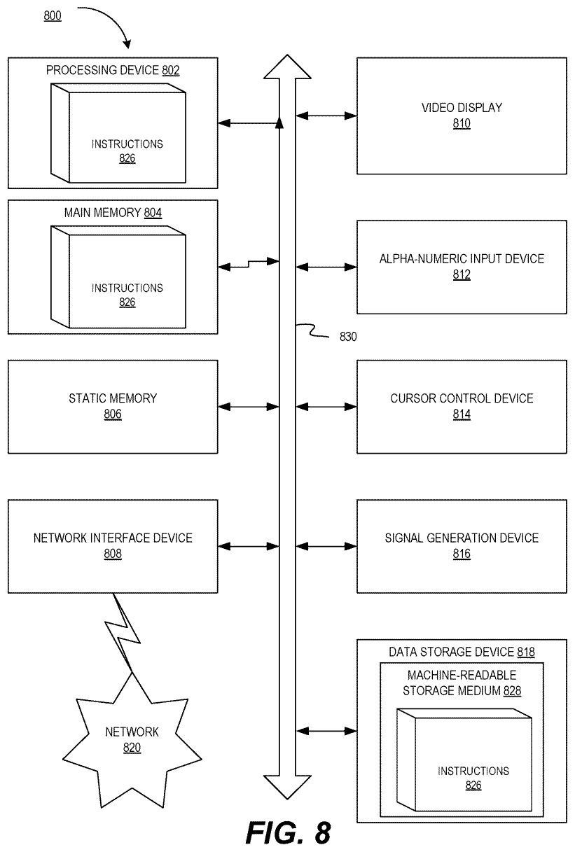

In some embodiments, the ultrasound system uses a simulator with pressure data to determine the grip map. In some embodiments, the simulator includes a processor coupled to a memory to input the pressure data and output the grip map. In some embodiments, the ultrasound system uses the grip maps for robotic assisted systems, telemedicine, and/or remote medicine. In some embodiments, the ultrasound system scores the quality of the grip as part of a sonographer certification. FIG. 8 is a block diagram of an example computing device 800 that can perform one or more of the operations described herein, in accordance with some embodiments. Computing device 800 can be connected to other computing devices in a LAN, an intranet, an extranet, and/or the Internet. The computing device can operate in the capacity of a server machine in client-server network environment or in the capacity of a client in a peer-to-peer network environment. The computing device can be provided by a personal computer (PC), a server computing, a desktop computer, a laptop computer, a tablet computer, a smartphone, or any machine capable of executing a set of instructions (sequential or otherwise) that specify actions to be taken by that machine. Further, while only a single computing device is illustrated, the term “computing device” shall also be taken to include any collection of computing devices that individually or jointly execute a set (or multiple sets) of instructions to perform the methods discussed herein. In some embodiments, the computing device 800 can be one or more of an access point and a packet forwarding component.

The example computing device 800 can include a processing device (e.g., a general-purpose processor, a PLD, etc.) 802, a main memory 804 (e.g., synchronous dynamic random-access memory (DRAM), read-only memory (ROM)), a static memory 806 (e.g., flash memory and a data storage device 818), which can communicate with each other via a bus 830. Processing device 802 can be provided by one or more general-purpose processing devices such as a microprocessor, central processing unit, or the like. In an illustrative example, processing device 802 can comprise a complex instruction set computing (CISC) microprocessor, reduced instruction set computing (RISC) microprocessor, very long instruction word (VLIW) microprocessor, or a processor implementing other instruction sets or processors implementing a combination of instruction sets. Processing device 802 can also comprise one or more special-purpose processing devices such as an application specific integrated circuit (ASIC), a field programmable gate array (FPGA), a digital signal processor (DSP), network processor, or the like. The processing device 802 can be configured to execute the operations described herein, in accordance with one or more aspects of the present disclosure, for performing the operations and steps discussed herein.

Computing device 800 can further include a network interface device 808 which can communicate with a network 820. The computing device 800 also can include a video display unit 810 (e.g., a liquid crystal display (LCD) or a cathode ray tube (CRT)), an alphanumeric input device 812 (e.g., a keyboard), a cursor control device 814 (e.g., a mouse) and an acoustic signal generation device 816 (e.g., a speaker, and/or a microphone). In one embodiment, video display unit 810, alphanumeric input device 812, and cursor control device 814 can be combined into a single component or device (e.g., an LCD touch screen).

Data storage device 818 can include a computer-readable storage medium 828 on which can be stored one or more sets of instructions 826, e.g., instructions for carrying out the operations described herein, in accordance with one or more aspects of the present disclosure. Instructions 826 can also reside, completely or at least partially, within main memory 804 and/or within processing device 802 during execution thereof by computing device 800, main memory 804 and processing device 802 also constituting computer-readable media. The instructions can further be transmitted or received over a network 820 via network interface device 808.

While computer-readable storage medium 828 is shown in an illustrative example to be a single medium, the term “computer-readable storage medium” should be taken to include a single medium or multiple media (e.g., a centralized or distributed database and/or associated caches and servers) that store the one or more sets of instructions. The term “computer-readable storage medium” shall also be taken to include any medium that is capable of storing, encoding or carrying a set of instructions for execution by the machine and that cause the machine to perform the methods described herein. The term “computer-readable storage medium” shall accordingly be taken to include, but not be limited to, solid-state memories, optical media and magnetic media.

Embodiments of automatically configuring of ultrasound systems based on a grip of an ultrasound scanner as described herein are advantageous, as they do not require manual, explicit configuration (e.g., setting imaging parameters) of the ultrasound system by an operator comparing to conventional ultrasound systems. An operator does not need to divert away from the patient and towards the ultrasound system that substantially improves the patient's care and reduces the cost.

Unless specifically stated otherwise, terms such as “transmitting,” “determining,” “receiving,” “generating,” “or the like, refer to actions and processes performed or implemented by computing devices that manipulates and transforms data represented as physical (electronic) quantities within the computing device's registers and memories into other data similarly represented as physical quantities within the computing device memories or registers or other such information storage, transmission or display devices. Also, the terms “first,” “second,” “third,” “fourth,” etc., as used herein are meant as labels to distinguish among different elements and can not necessarily have an ordinal meaning according to their numerical designation.

Examples described herein also relate to an apparatus for performing the operations described herein. This apparatus can be specially constructed for the required purposes, or it can comprise a general-purpose computing device selectively programmed by a computer program stored in the computing device. Such a computer program can be stored in a computer-readable non-transitory storage medium.

The methods and illustrative examples described herein are not inherently related to any particular computer or other apparatus. Various general-purpose systems can be used in accordance with the teachings described herein, or it can prove convenient to construct more specialized apparatus to perform the required method steps. The required structure for a variety of these systems will appear as set forth in the description above. The above description is intended to be illustrative, and not restrictive. Although the present disclosure has been described with references to specific illustrative examples, it will be recognized that the present disclosure is not limited to the examples described. The scope of the disclosure should be determined with reference to the following claims, along with the full scope of equivalents to which the claims are entitled.

As used herein, the singular forms “a”, “an” and “the” are intended to include the plural forms as well, unless the context clearly indicates otherwise. It will be further understood that the terms “comprises”, “comprising”, “includes”, and/or “including”, when used herein, specify the presence of stated features, integers, steps, operations, elements, and/or components, but do not preclude the presence or addition of one or more other features, integers, steps, operations, elements, components, and/or groups thereof. Therefore, the terminology used herein is for the purpose of describing particular embodiments only and is not intended to be limiting.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Although the method operations were described in a specific order, it should be understood that other operations may be performed in between described operations, described operations may be adjusted so that they occur at slightly different times or the described operations may be distributed in a system which allows the occurrence of the processing operations at various intervals associated with the processing.

Various units, circuits, or other components may be described or claimed as “configured to” or “configurable to” perform a task or tasks. In such contexts, the phrase “configured to” or “configurable to” is used to connote structure by indicating that the units/circuits/components include structure (e.g., circuitry) that performs the task or tasks during operation. As such, the unit/circuit/component can be said to be configured to perform the task, or configurable to perform the task, even when the specified unit/circuit/component is not currently operational (e.g., is not on). The units/circuits/components used with the “configured to” or “configurable to” language include hardware—for example, circuits, memory storing program instructions executable to implement the operation, etc. Reciting that a unit/circuit/component is “configured to” perform one or more tasks, or is “configurable to” perform one or more tasks, is expressly intended not to invoke 35 U.S.C. 112, sixth paragraph, for that unit/circuit/component. Additionally, “configured to” or “configurable to” can include generic structure (e.g., generic circuitry) that is manipulated by software and/or firmware (e.g., an FPGA or a general-purpose processor executing software) to operate in manner that is capable of performing the task(s) at issue. “Configured to” may also include adapting a manufacturing process (e.g., a semiconductor fabrication facility) to fabricate devices (e.g., integrated circuits) that are adapted to implement or perform one or more tasks. “Configurable to” is expressly intended not to apply to blank media, an unprogrammed processor or unprogrammed generic computer, or an unprogrammed programmable logic device, programmable gate array, or other unprogrammed device, unless accompanied by programmed media that confers the ability to the unprogrammed device to be configured to perform the disclosed function(s).

The foregoing description, for the purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the embodiments and its practical applications, to thereby enable others skilled in the art to best utilize the embodiments and various modifications as may be suited to the particular use contemplated. Accordingly, the present embodiments are to be considered as illustrative and not restrictive, and the invention is not to be limited to the details given herein, but may be modified within the scope and equivalents of the appended claims.

What is claimed is:

1. An ultrasound system comprising:
an ultrasound scanner having a touch sensitive surface; and
a processor configured to:
determine a grip orientation on the touch sensitive surface; and
activate, based on the grip orientation, a region of the touch sensitive surface to accept a user input.

2. The ultrasound system as described in claim 1, wherein the processor is implemented to deactivate the region of the touch sensitive surface to accept the user input.

3. The ultrasound system as described in claim 1, wherein the processor is implemented to deactivate, based on the grip orientation, an additional region of the touch sensitive surface to accept the user input.

4. The ultrasound system as described in claim 1, further comprising a display device implemented to display a visual representation of the region of the touch sensitive surface.

5. The ultrasound system as described in claim 4, wherein the visual representation indicates a functionality for the user input.

6. The ultrasound system as described in claim 1, wherein the processor is implemented to activate the region to accept the user input as a swiping gesture that controls at least one of a gain and a depth, and the ultrasound scanner is implemented to generate ultrasound signals based on the at least one of the gain and the depth.

7. The ultrasound system as described in claim 1, further comprising a display device implemented to display an ultrasound image based on ultrasound data generated by the ultrasound scanner, wherein the processor is implemented to adjust a zoom level of the ultrasound image based on a pressure of the user input on the region of the touch sensitive surface.

8. The ultrasound system as described in claim 1, wherein the ultrasound scanner includes at least one light source, and the processor is implemented to activate the at least one light source to emit light to indicate the region of the touch sensitive surface.

9. The ultrasound system as described in claim 1, wherein the grip orientation includes at least one finger location, and the region is proximate to, and disjoint from, the at least one finger location.

10. An ultrasound system comprising:
an ultrasound scanner having a touch sensitive surface and configured to generate ultrasound data based on reflections of ultrasound signals transmitted by the ultrasound scanner at an anatomy;
a display device configured to generate an ultrasound image of the anatomy based on the ultrasound data; and
a processor configured to:
determine locations of pressure on the touch sensitive surface and amounts of the pressure at the locations; and
determine, based on the locations and the amounts of the pressure and the ultrasound image, an elasticity of the anatomy.

11. The ultrasound system as described in claim 10, wherein the processor is implemented to determine, based on the elasticity, a classification of the anatomy as at least one of a vein and an artery.

12. The ultrasound system as described in claim 10, wherein the touch sensitive surface is excluded from a lens surface of the ultrasound scanner, and the processor is implemented to determine the amounts of the pressure in a direction towards the lens surface.

13. The ultrasound system as described in claim 10, wherein the touch sensitive surface includes a lens surface of the ultrasound scanner, and at least some of the locations of the pressure are on the lens surface.

14. The ultrasound system as described in claim 10, wherein the processor is implemented to determine that the amounts of the pressure correspond to an excessive pressure, and the display device is implemented to display a warning that indicates the excessive pressure.

15. The ultrasound system as described in claim 10, wherein the processor is implemented to:
determine, based on at least one of the locations and the amounts of the pressure, an amount of uncoupling of the ultrasound scanner from a patient; and
adjust, based on the amount of uncoupling, at least one imaging parameter.

16. An ultrasound system comprising:
an ultrasound scanner having a touch sensitive surface and configured to generate ultrasound data based on reflections of ultrasound signals transmitted by the ultrasound scanner;
a display device configured to display an ultrasound image that is based on the ultrasound data; and
a processor configured to:
determine a grip orientation on the touch sensitive surface; and
set, based on the grip orientation, an imaging parameter for at least one of the display device and the ultrasound scanner.

17. The ultrasound system as described in claim 16, wherein the processor is implemented to set the imaging parameter to control beamforming of at least one of the ultrasound signals and the reflections.

18. The ultrasound system as described in claim 16, wherein the processor is implemented to determine, based on the grip orientation, a patient anatomy, and set the imaging parameter based on the patient anatomy.

19. The ultrasound system as described in claim 16, wherein the imaging parameter includes at least one of a depth and a gain for the ultrasound signals transmitted by the ultrasound scanner.

20. The ultrasound system as described in claim 16, wherein the ultrasound scanner includes an inertial measurement unit configured to generate orientation data, and the processor is implemented to determine, based on the orientation data, an orientation of the ultrasound scanner; and
further comprising a neural network implemented to determine the imaging parameter based on the grip orientation and at least one of the ultrasound image, the orientation of the ultrasound scanner, an operator identification, and a voice command.
